(19) United States
(12) Patent Application Publication
(10) Pub. No.: US 2009/0182198 A1
(43) Pub. Date: Jul. 16, 2009

(54) MULTIPLE BAND DISPENSER ENDOSCOPE SHEATH

(76) Inventors: Gregory J. Skerven, Kernersville, NC (US); William S. Gibbons, Winston-Salem, NC (US)

Correspondence Address: BRINKS HOFER GILSON & LIONE/CHICAGO/COOK, PO BOX 10395, CHICAGO, IL, 60610, US

(21) Appl. No.: 12/343,010

(22) Filed: Dec. 23, 2008

Related U.S. Application Data
(60) Provisional application No. 61/009,359, filed on Dec. 27, 2007.

(51) Int. Cl. A61B 1/00

(52) U.S. Cl. 600/121

(57) ABSTRACT

A protective sheath comprising a cap member configured for attachment to the distal end of an endoscope and a flexible, elongate outer sleeve attached to the cap member and adapted to unfold over the endoscope in a proximal direction along a substantial portion of the endoscope to protect the endoscope from bodily fluids.

### TECHNICAL FIELD

The present invention relates to a protective endoscope sheath with a functional cap member.

### BACKGROUND OF THE INVENTION

Endoscopes are expensive devices critical in the field of diagnostic and therapeutic indications. An endoscope may typically be used in a surgical procedure for a brief period of time. However, after it is used, the device must be meticulously cleaned. Not only must the surface of the endoscope be cleaned, but also any channels used for air, water, biopsy, or suction. In fact, any endoscope surface or channel that comes into contact with bodily fluids must be cleaned after each use. Such cleaning decreases the amount of time the endoscope can be used in surgery and also increases the risk that the endoscope may be damaged during cleaning.

Protective endoscope sheaths were developed to eliminate or significantly decrease the cleaning time required for endoscopes. Most of these sheaths roll onto an endoscope similar to a condom. In some instances, the endoscope sheaths extend from the endoscope cap to the control mechanism at the endoscope's proximate end.

There are a variety of devices used in conjunction with endoscopes that attach to the endoscope at various locations. One such device is a band ligator. Ligators include designs, such as the multiple band ligator (MBL) or single band ligator, that individually tether or otherwise secure the bands to the ligator and then release them sequentially as needed, often by use of one or more strings extending to the proximal end. Ligation is often used in procedures such as polypectomy and Endoscopic Mucosal Resection (EMR). Polypectomy is a procedure for the removal of polyps and EMR is a medical procedure used to remove tissue during endoscopy. Ligation is often used along with a surgical snare in EMR.

These procedures can be complicated by protective sheaths. An activation line typically extends from the ligator through the operating channel of the endoscope. In EMR, for example, the surgical snare can be placed in the endoscope operating channel along with an activation line that connects the ligator to an activating component located at the proximal end of the endoscope. Using both tools is often difficult or prohibited when a protective sheath is used as the operating channel is covered by the sheath. In some instances, the operating channel may be too small to accommodate both the surgical snare and the activation line. On the outside of the endoscope, the ligating barrel may be difficult to secure to the scope if the scope has a protective sheath on it.

Thus, there is a need for an endoscopic system where using multiple tools such as an MBL or a surgical snare is not prohibited or hampered by a protective sheath. There is also a need to provide a protective sheath that assists in securing an MBL barrel to an endoscope.

### BRIEF SUMMARY

The present invention provides a sheath for use with an endoscope that comprises a cap member configured to be attached at the distal end of an endoscope and a flexible, elongate outer sleeve attached to the cap member. The outer sleeve is adapted to unfold over an exterior surface of the endoscope in a proximal direction along a substantial portion of the endoscope to protect the endoscope from internal bodily fluids and to secure the cap member to the endoscope.

In yet another aspect of the present invention, there is a method of attaching to an endoscope a sheath comprising a flexible, elongated outer sleeve, an elongated inner sleeve, and a distal end portion connecting the outer sleeve with the inner sleeve. The method comprises inserting the inner sleeve into an access channel of an endoscope and attaching the outer sleeve over an exterior surface of the endoscope by unfolding the outer sleeve in a proximal direction along a substantial portion of the endoscope to protect the endoscope from internal bodily fluids.

These and other features of the invention will become apparent upon review of the following detailed description of the presently preferred embodiments of the invention, taken into conjunction with the appended figures.

### BRIEF DESCRIPTION OF THE DRAWINGS

FIG. 1 is a profile view of the sheath placed onto an endoscope before being unfolded over the endoscope.

FIG. 2 is a profile view of the outer sleeve of the sheath unfolded over the endoscope.

FIG. 3A is a profile view of the sheath of the present invention protecting an endoscope and an inner sleeve inserted into an accessory channel in the endoscope. FIG. 3B is view of the distal end of the cap member.

FIG. 4 shows an embodiment where a secondary lumen extends through the outer sleeve and in the lumen of the outer sleeve.

FIG. 5 shows an embodiment where a secondary lumen extends through the outer sleeve and outside the lumen of the outer sleeve.

FIG. 6 is a perspective view of the sheath covering an endoscope with ligating barrel.

### DETAILED DESCRIPTION OF THE DRAWINGS AND THE PRESENTLY PREFERRED EMBODIMENTS

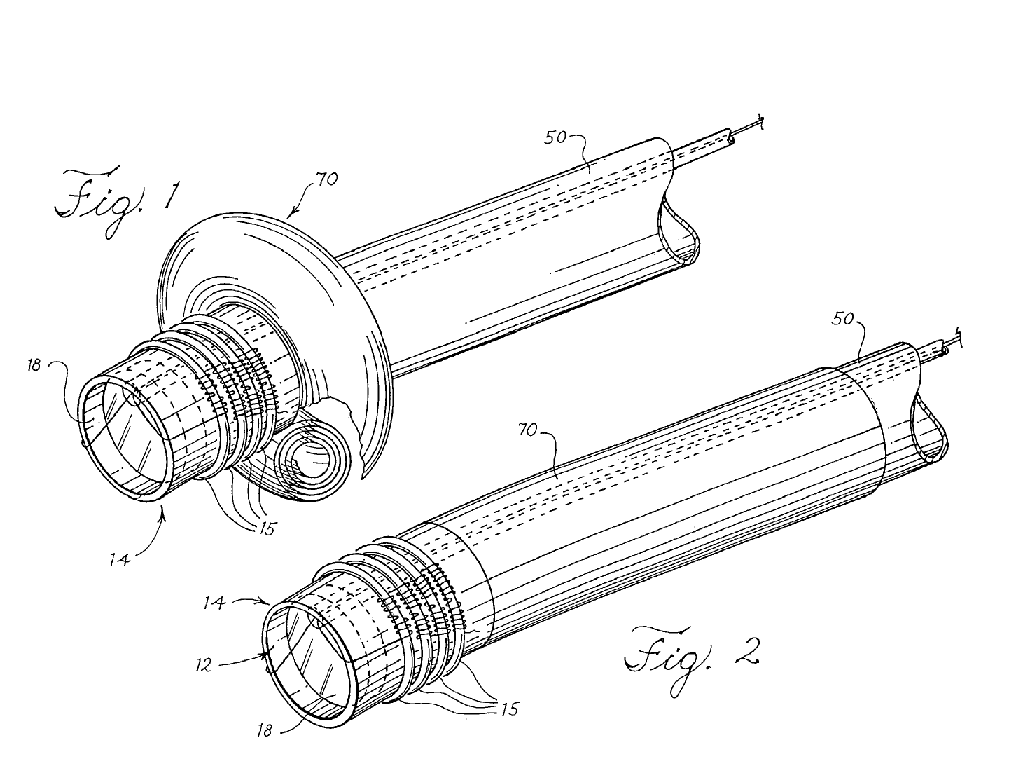

The present invention provides a protective sheath for an endoscope 50. The sheath comes in a folded state before deployment over an endoscope. FIG. 1 shows an embodiment of the sheath initially being placed on the distal end of an endoscope 50. The sheath comprises a cap member 14 that attaches to the distal end of an endoscope 50 and a flexible, elongate outer sleeve 70 attached to the cap member 14. The cap member is tubular having a proximal end engaging the distal end of the elongate outer sleeve 70 and defines a cavity 12 adjacent to a plate 18 that is adjacent to the distal end of the endoscope. The proximal end of the cavity 12 is bordered by the plate 18 that is adjacent to the distal end of the endoscope. In embodiments used in ligation, body tissue is drawn into the cavity 12 once a vacuum is applied.

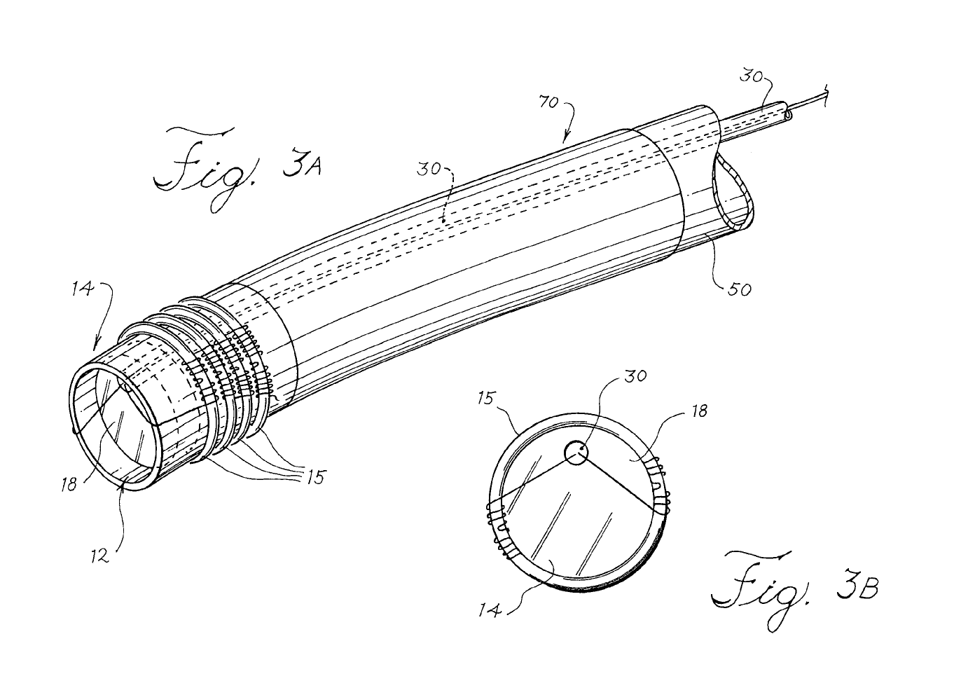

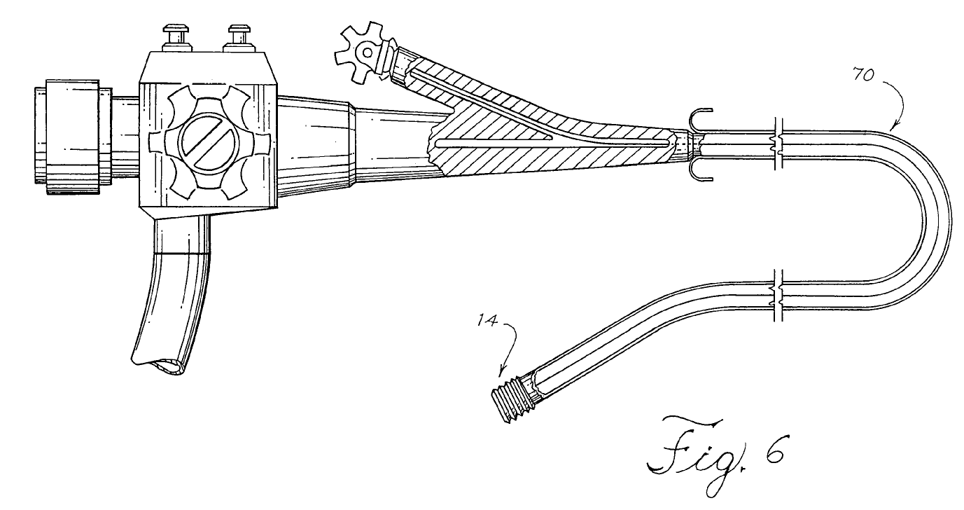

The cap member 14 may be functional in that it is capable of supporting endoscopic devices used in ligation, such as the ligating sheath shown in FIG. 1 that comprises a ligating barrel. In other embodiments, the cap member 14 is any endoscopic device attachable to the distal end of an endoscope. The flexible, elongate outer sleeve 70 is adapted to unfold over an exterior surface 55 of the endoscope 50 in a proximal direction along a substantial portion of the endoscope 50 to protect it from internal bodily fluids and to secure the cap member 14 to the endoscope 50. In some embodiments, the outer sleeve 70 is in an unrolled or unfolded state and is pulled over an endoscope 50. FIG. 2 shows the sheath unfolded over a portion of the endoscope 50. FIG. 6 shows the protective sheath 10 unfurled over the length on an endoscope 50.

The sheath is preferably made of a hermetically sealable material such as a polymer or a fabric material having high elasticity such that the outer sleeve 70 can be unfolded when added to the endoscope 50 and flexible enough to move with an endoscope 50 when in use. The resin can be tetrafluoroethylene, polyethylene, or the like. The sheath can be made of an elastomeric material, for example, polyurethane-based elastomer, polyester-based elastomer, polyolefine-based elastomer, polyamide-based elastomer, polystyrene-based elastomer, fluorine-based elastomer, silicone rubber, fluororubber, latex rubber, and the like. They can be used alone or as a mixture of two or more thereof. The outer sleeve 70 is preferably elastic to grip the endoscope.

The outer sleeve 70 has a range of widths and lengths that depends on the size of the endoscope to be used. In some embodiments the lengths can be from about up to about 100 cm to about 200 cm or any combination or subcombination therein. A length that may be preferred is about 100 cm to about 110 cm. In some embodiments, a sheath has a wall thickness of between about 2 mm to about 8 mm or any combination or subcombination therein. In another embodiment, a sheath has a wall thickness of between about 4 mm to about 6 mm.

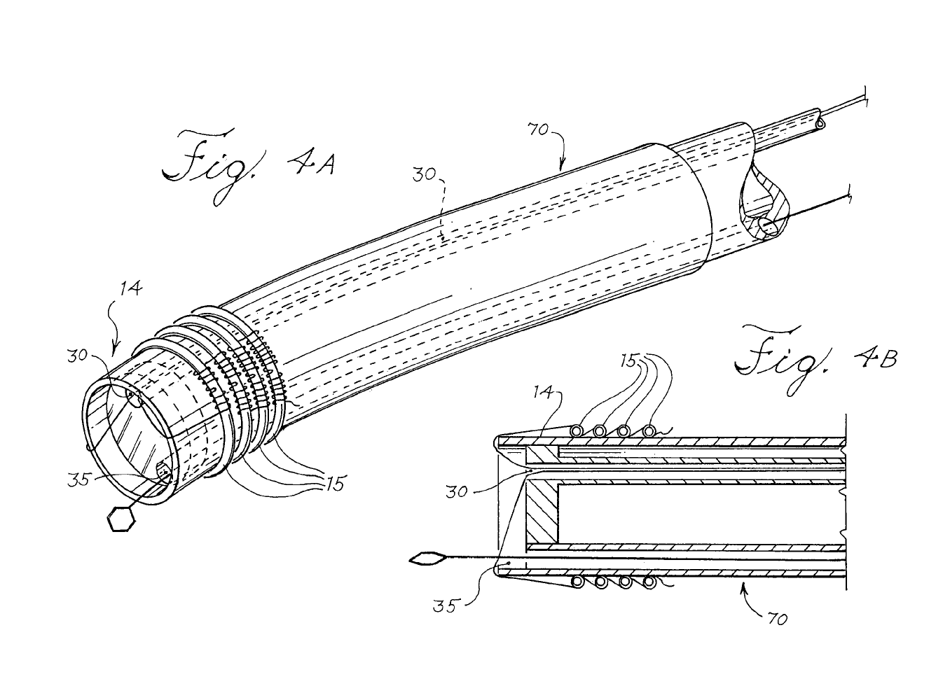

In some embodiments, the sheath further comprises an elongate inner sleeve 30, as shown in FIGS. 3A and 3B. The elongate inner member 30 has an inner lumen therethrough that meets the plate 12 at the distal end of the inner member 30. It is adapted to fit within an accessory channel of the endoscope 50. The inner member is attached to the plate 12 to provide a hermetic seal. There may be more than one inner member 30 depending on the number of accessory channels available in the endoscope being used. The inner member 30 protects the accessory channel from bodily fluids during a procedure. The accessory channel, therefore, does not have to undergo a time consuming cleaning process after use. The elongate inner member 30 is preferably more rigid than the elongate outer sleeve 70 so that an operator can easily insert the inner member 30 into the accessory channel of an endoscope 50. The walls of the inner member 30 can be 1 mm to about 3 mm thick (or any combination or subcombination therein) and made of a stiff resin of tetrafluoroethylene, polyethylene, or the like. The inner member 30 can extend along the accessory channel to the proximal end of the endoscope 50 in preferred embodiments.

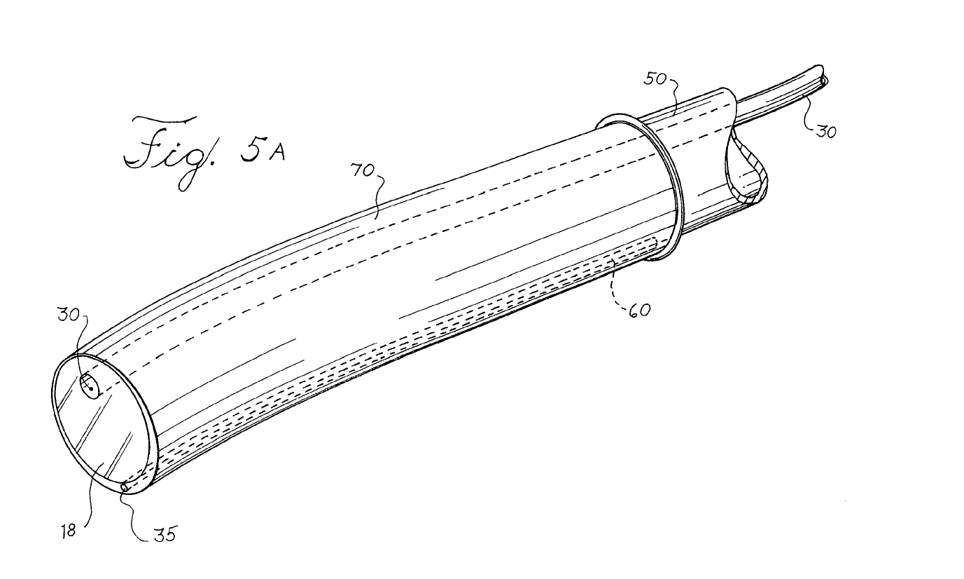

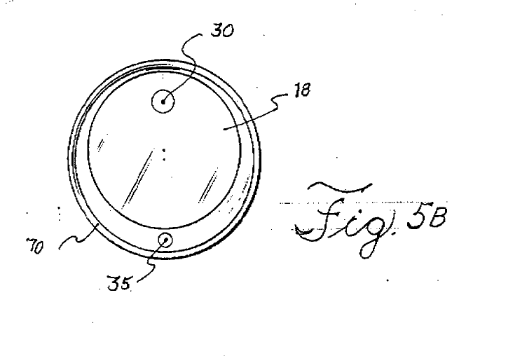

Because of the presence of the inner member 30 in the accessory channel, the accessible area in the accessory channel is decreased. As a consequence, the sheath may be provided with a secondary lumen 60, which provides another channel for medical instrument use as shown in FIGS. 4, 5A, and 5B. The sheaths of some embodiments of the present invention have at east one secondary lumen 60 extending through the outer sleeve 70 with a secondary opening 35 in the cap member 14. The cap member 14 connects the outer sleeve 70 with the inner member 30. The secondary lumen 60 has a distal secondary opening 35 in the cap member 14 and on the plate 18, with a proximal opening at the proximate end of the sheath. The secondary lumen 60 can contain an activation line disposed therethrough and operably connected to the ligating barrel. The secondary lumen 60 also can comprise an electrical snare disposed therethrough or can be used to apply suction for ligation procedures. The secondary lumen 60 can also be a vacuum channel.

In embodiments with an activation line, the line is threaded through the secondary lumen 60 with one end of the line attached to a ligating barrel though the distal opening 35 and another end attached to an actuation device at the proximal end of the secondary lumen 60. In some embodiments, the secondary lumen 60 can be inside the lumen of the outer sleeve 70 or outside the outer sleeve 70 lumen as shown in FIG. 5.

In some embodiments, the cap member 14 is a ligating barrel. The ligating barrel 14 is attached to the sheath to provide a unitary instrument. As shown in FIGS. 1, 2, 3A, 4A, and 6, the protective system has a ligating barrel 14 attached to the distal end 40 of the sheath 10. A more detailed description of barrel 14 can be found in U.S. Pat. No. 5,624,453, incorporated herein by reference in its entirety. The cap member is preferably made of a transparent plastic material or some other inert material such as stainless steel. The cap member 14 is integrally attached to the elongate member 70 to provide a tight friction fit over the distal end 40 of the endoscope. In some embodiments, the cap member 14 is removable from the elongate member 70 leaving only the plate 18 at the distal end as shown in FIG. 5A.

The ligating barrel 14 is operably connected to an activation line. In some embodiments, the ligating barrel 14 comprises one or more ligating bands 15 removably disposed on the ligating barrel 14 and coupled to an activating component via the activation line, wherein operation of the activating component releases one or more ligating bands 15 from the ligating barrel 14.

The sheath is adapted to protect current endoscope systems and to allow the same level of useability. As such, the inner member 30 is adapted to allow passage for a medical instrument, such as an electrosurgical snare. In embodiments that may be preferred, the sheath is adapted to work with a surgical snaring system that comprises a snare made from braided stainless steel cable with a hexagonal shape when in the open configuration. A suitable surgical snare system is the 7FR Soft AcuSnare Mini Hexagonal Head disposable polypectomy snare, sold by Wilson-Cook Medical Inc., catalog no. SASMH-1.

Herein provided is also a method of attaching to an endoscope 50 a sheath as described above comprising inserting an inner member 30 into an access channel of an endoscope 50 and attaching the outer sleeve 70 over an exterior surface of the endoscope 50 by unfolding the outer sleeve 70 in a proximal direction along a substantial portion of the endoscope 50 to protect the endoscope 50 from internal bodily fluids. The sheath, in some embodiments, comprises a flexible, elongated outer sleeve 70, an elongated inner member 30, and a distal end portion 24 connecting the outer sleeve 70 with the inner member 30.

The inner member 30 can be inserted along a substantial portion of the access channel. The method further comprises mating the cap member 14 with the distal end of the endoscope 50. In such embodiments, the cap member 14 of the sheath is transparent. In other embodiments, the cap member 14 is a ligating barrel as described herein. When a ligating barrel is used, the method further comprises connecting the ligating barrel to an activation line. In some embodiments, the activation line extends through the secondary lumen 60 to an activation device.

It will, of course, be well understood from the discussions above that other known ligating barrel designs, activation mechanisms, endoscope systems, etc. could be used within the scope of the invention. It is therefore intended that the foregoing detailed description be regarded as illustrative rather than limiting, and that it be understood that it is the following claims, including all equivalents, that are intended to define the spirit and scope of this invention.

1. A sheath for protecting an endoscope, the sheath comprising:
a cap member that attaches to the distal end of the endoscope;
a flexible, elongate outer sleeve coupled to the cap member, and unfolds over an exterior surface of the endoscope in a proximal direction along a substantial portion of the endoscope to protect the endoscope from internal bodily fluids and to secure the cap member to the endoscope; and
wherein the cap member is tubular with a proximal end engaging the distal end of the flexible elongate outer sleeve and defines a cavity adjacent to a plate that is adjacent to the distal end of the endoscope.

2. The sheath of claim 1 further comprising at least one elongate inner member coupled to the cap member and fits within an accessory channel of the endoscope, the at least one inner member comprising a lumen therethrough.

3. The sheath of claim 1 wherein the cap member is a ligating barrel.

4. The sheath of claim 1 wherein the cap member is attached to the distal end by a frictional fit.

5. The sheath of claim 4 wherein the ligating barrel comprises one or more ligating bands removably disposed on the ligating barrel and the ligating barrel is coupled to an activating component via the activation line.

6. The sheath of claim 2 wherein the inner member holds a medical instrument.

7. The sheath of claim 6 wherein the medical instrument is an electrosurgical snare.

8. The sheath of claim 1 wherein the outer sleeve unfolds in a proximate direction over an entire endoscope.

9. The sheath of claim 1 further comprising at least one secondary lumen extending through the outer sleeve.

10. The sheath of claim 9 wherein the at least one secondary lumen is a vacuum channel.

11. A method of attaching to an endoscope a sheath comprising a flexible, elongated outer sleeve; an elongated inner member; and a distal end portion connecting the outer sleeve with the inner member; the method comprising:
inserting the inner member into an access channel of an endoscope; and
attaching the outer sleeve over an exterior surface of the endoscope by unfolding the outer sleeve in a proximal direction along a substantial portion of the endoscope to protect the endoscope from internal bodily fluids.

12. The method of claim 11 further comprising mating the distal end portion with a distal end of the endoscope.

13. The method of claim 11 wherein the distal end portion is transparent.

14. The method of claim 11 comprising inserting the inner member along a substantial portion of the access channel.

15. The method of claim 11 further comprising at least one lumen extending through the outer sleeve.

16. The method of claim 15 wherein the at least one lumen extending through the outer sleeve is a vacuum channel.

17. The method of claim 11 further comprising a ligating barrel on the distal end portion of the sheath.

18. The method of claim 17 further comprising operably connecting the ligating barrel to an activation line.

19. The method of claim 18 further comprising at least one lumen extending through the outer sleeve comprising the activation line.

20. The method of claim 17 wherein the ligating barrel comprises one or more ligating bands removably disposed on the ligating barrel and coupled to an activating component via the activation line, wherein operation of the activating component releases one or more ligating bands from the ligating barrel.

21. A protective ligating sheath comprising:
a ligating barrel that attaches to the distal end of an endoscope with one or more ligating bands removably disposed on the ligating barrel; and
a flexible, elongate outer sleeve coupled to the ligating barrel, and unfolds over an exterior surface of the endoscope in a proximal direction along a substantial portion of the endoscope to protect the endoscope from internal bodily fluids and to secure the ligating barrel to the endoscope.

22. The protective ligating sheath of claim 21 further comprising at least one elongate inner member coupled to the ligating barrel and fits within an accessory channel of the endoscope, the at least one inner member comprising a lumen therethrough.

23. The protective ligating sheath of claim 21 wherein the ligating barrel attaches to the distal end of the endoscope by a frictional fit.

24. The protective ligating sheath of claim 21 wherein the ligating barrel is coupled to an activating component via the activation line.

25. The protective ligating sheath of claim 21 wherein the inner member holds a medical instrument.

26. The protective ligating sheath of claim 25 wherein the medical instrument is an electrosurgical snare.

27. The protective ligating sheath of claim 1 wherein the outer sleeve unfolds in a proximate direction over the entire endoscope.

28. The protective ligating sheath of claim 21 further comprising at least one secondary lumen extending through the outer sleeve.
